(19) United States
(12) Patent Application Publication
(10) Pub. No.: US 2020/0214606 A1
(43) Pub. Date: Jul. 9, 2020

(54) SIMPLE SUGAR CONCENTRATION SENSOR AND METHOD WITH NARROWED OPTICAL PATH AND INTERROGATOR BEAM

(71) Applicant: K Sciences GP, LLC, Huntsville, AL (US)

(72) Inventor: Valentin Korman, Huntsville, AL (US)

(73) Assignee: K Sciences GP, LLC, Huntsville, AL (US)

(21) Appl. No.: 16/700,154

(22) Filed: Dec. 2, 2019

Related U.S. Application Data
(60) Provisional application No. 62/788,587, filed on Jan. 4, 2019.

(51) Int. Cl. A61B 5/145; A61B 5/1455

(52) U.S. Cl. CPC A61B 5/14532; A61B 5/14558

(57) ABSTRACT

A glucose sensor comprising one or more optical energy source is disclosed. In various embodiments one or more beam splitter/combiner is implemented to receive optical energy from the one or more optical energy source. One or more beam splitter/combiner provides provide optical energy to detector(s) to determine glucose in a body tissue.

### CROSS-REFERENCE TO RELATED APPLICATION(S)

This application claims priority to and the benefit of U.S. Prov. Pat. App. No. 62/788,587 entitled “Simple Sugar Concentration Sensor and Method with Narrowed Optical Path and Interrogator Beam,” filed on Jan. 4, 2019, the contents of which are hereby incorporated herein by reference in their entirety for any purpose.

### FIELD

The present invention relates to monitoring of simple sugar (or monosaccharide) content within a fluid. More specifically, the invention uses an optical energy source in combination with polarizers to determine the change in a sugar level (e.g., glucose) of a subject fluid relative to a baseline concentration, such as blood.

### BACKGROUND

Simple sugar changes the polarization of the optical energy passing through it according to the equation Θ=α×L×C, where L is the travel length of the energy through the fluid in which the sugar is concentrated, C is the sugar concentration, and α is a constant that depends on the type of sugar, wavelength of the energy, and the fluid. If L and a are known, by measuring the change in polarization of energy passing through a sugar-containing fluid relative to a baseline measurement, the sugar concentration of the fluid can be derived.

This principle may be used, for example, to non-invasively determine the glucose concentration of human blood. Normal blood has a non-zero glucose concentration C, which causes a change in polarization for energy passing through the blood. For a glucose concentration of 70 mg/dL and an α=45.62 (×10⁻⁶) degrees/mm/(mg/dL), energy of wavelength 633 nm and a 3.0 mm path length will have a rotation Θ of 0.00958 degrees. Measuring the change in rotation caused by the sugar allows derivation of the current sugar concentration.

### SUMMARY

A non-invasive system for measuring glucose is provided. The system may include a first light source emitting a light capable of penetrating a body tissue. There may be a beam splitter/combiner to receive the light. A first detector may be included and may be optically coupled to the beam splitter/combiner. A second detector may be optically coupled to the beam splitter/combiner. The first detector and the second detector may be operated to measure glucose in the body tissue in response to the light.

The first detector and the second detector may be operated to measure glucose in the body tissue by calculating a difference in amplitude of the light detected by the first detector and the light detected by the second detector. At least one of the first detector and the second detector may include a polarizer.

In various embodiments, the system has a first polarizer proximal to the first light source for receiving at least a portion of the light emitted from the first light source and for providing a first polarized light, and a second polarizer to receive at least a portion of the first polarized light following passage of the first polarized light through the body tissue and to provide a second polarized light. The first detector may be positioned in a manner to detect at least a portion of the first polarized light via the beam splitter/combiner. The second detector may be positioned in a manner to detect at least a portion of the second polarized light via the beam splitter/combiner. At least one of the first detector and the second detector may determine a relative intensity of at least one of the first polarized light and the second polarized light.

In some embodiments of the non-invasive system, a second light source is provided. The second light source may emit a second light capable of penetrating the body tissue, the first light source and the second light source both providing light to a second beam splitter/combiner. The second beam splitter/combiner may combine light from both the first light source and the second light source for provision to the body tissue.

In various instances, the first light source is a source of collimated light. In various instances, the first light source is a source of collimated light and the second light source is a source of non-collimated light. Furthermore, in various instances, the first light source is a laser.

A further non-invasive system for measuring glucose is provided. The system may have a first light source emitting a first light, a polarizer configured to receive the first light and polarize the first light, the polarizer emitting a second light capable of penetrating a body tissue, the second light made up of polarized first light, and a first beam splitter/combiner to receive the second light following penetration into, through, and out of the body tissue. There may be a first detector optically coupled to the first beam splitter/combiner to receive the second light, a second polarizer optically coupled to the first beam splitter/combiner to receive the second light and emit a third light made up of a further polarized second light, and a second detector optically coupled to the second polarizer to receive the third light. The first detector and the second detector may be operated to measure glucose in the body tissue by comparing an intensity of the second light and an intensity of the third light.

In various embodiments, the system has a second light source emitting a fourth light and a second beam splitter/combiner to receive the second light and the fourth light and provide the second light and the fourth light to the body tissue for penetration into the body tissue. The first beam splitter/combiner further receives the fourth light following penetration into, through, and out of the body tissue.

Moreover, the first detector may be optically coupled to the beam splitter/combiner to further receive the fourth light. The second and/or first detector performs a calibration in response to the fourth light. The first light source and the second light source may emit light simultaneously. The first light source and the second light source may emit light simultaneously wherein the first light source emits the first light having a first center frequency and the second light source emits the fourth light having a second frequency, the first center frequency and the second center frequency being different frequencies. Furthermore, the first light source may emit first light that is pulsed. The first light source may emit first light that is modulated by a first modulation and wherein at least one of the first detector and the second detector detects the first modulation. The first light source may emit collimated light and the second light source may emit non-collimated light. The first light source may emit non-collimated light and the second light source may emit collimated light.

A method is also provided. A method of non-invasive glucose measurement may include providing a first light source emitting a light capable of penetrating a body tissue and providing a beam splitter/combiner to receive the light. The method may include providing a first detector optically coupled to the beam splitter/combiner, and providing a second detector optically coupled to the beam splitter/combiner. In various instances, the first detector and the second detector are operated to measure glucose in the body tissue.

The method may include further aspects. For example, the method may include providing a first polarizer proximal to the first light source for receiving at least a portion of the light emitted from the first light source and for providing a first polarized light. The method may include providing a second polarizer to receive at least a portion of the first polarized light following passage of the first polarized light through the body tissue and to provide a second polarized light. In various embodiments, the first detector is positioned in a manner to detect at least a portion of the first polarized light via the beam splitter/combiner, and the second detector is positioned in a manner to detect at least a portion of the second polarized light via the beam splitter/combiner. In various embodiments, at least one of the first detector and the second detector determines a relative intensity of at least one of the first polarized light and the second polarized light.

The method may also include providing a polarizer with at least one of the first detector and the second detector. Moreover, the first detector and the second detector may be operable to measure glucose in the body tissue by calculating a difference in amplitude of the light detected by the first detector and the light detected by the second detector.

### BRIEF DESCRIPTION OF THE DRAWINGS

FIG. 1A is a system diagram of an embodiment of the invention.

FIG. 1B is a system diagram of an embodiment of the invention including a feedback aspect.

FIG. 2A is the system diagram of FIG. 1A showing the embodiment in use with a human ear.

FIG. 2B is the system diagram of FIG. 1B showing the embodiment in use with a human ear.

FIG. 3 is an optical tissue model of a human ear;

FIG. 4 is an optical tissue model of a human ear depicting various optical paths of relatively similar characteristics;

FIG. 5 is an optical tissue model of a human ear depicting various optical paths of relatively dissimilar characteristics;

FIG. 6 depicts an example embodiment of a simple sugar concentration sensor and method with a narrowed optical path, in accordance with various embodiments; and

FIG. 7 depicts an example embodiment of a simple sugar concentration sensor and method with a narrowed optical path and further including an interrogator beam, in accordance with various embodiments.

### DETAILED DESCRIPTION

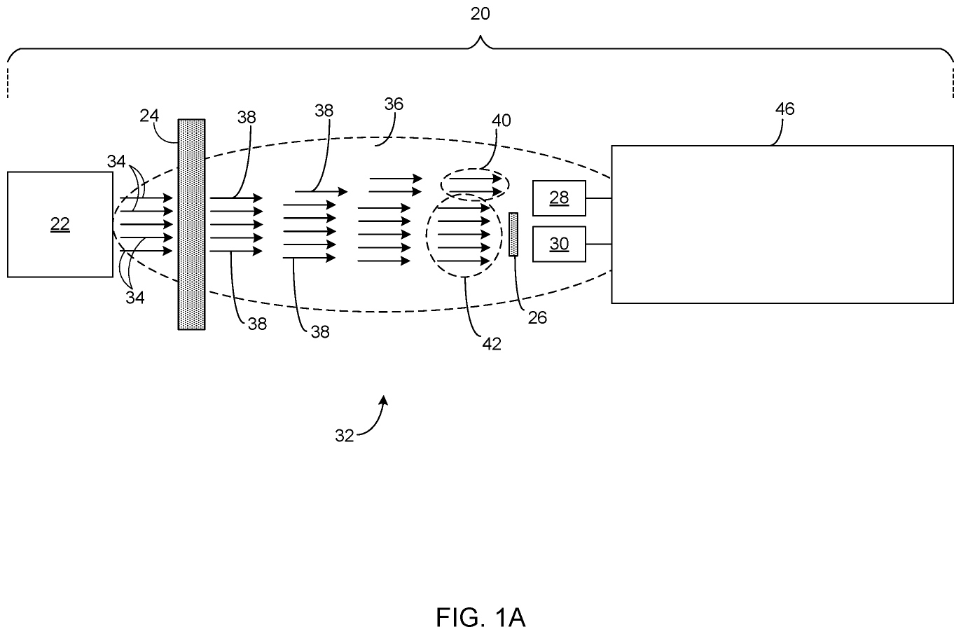

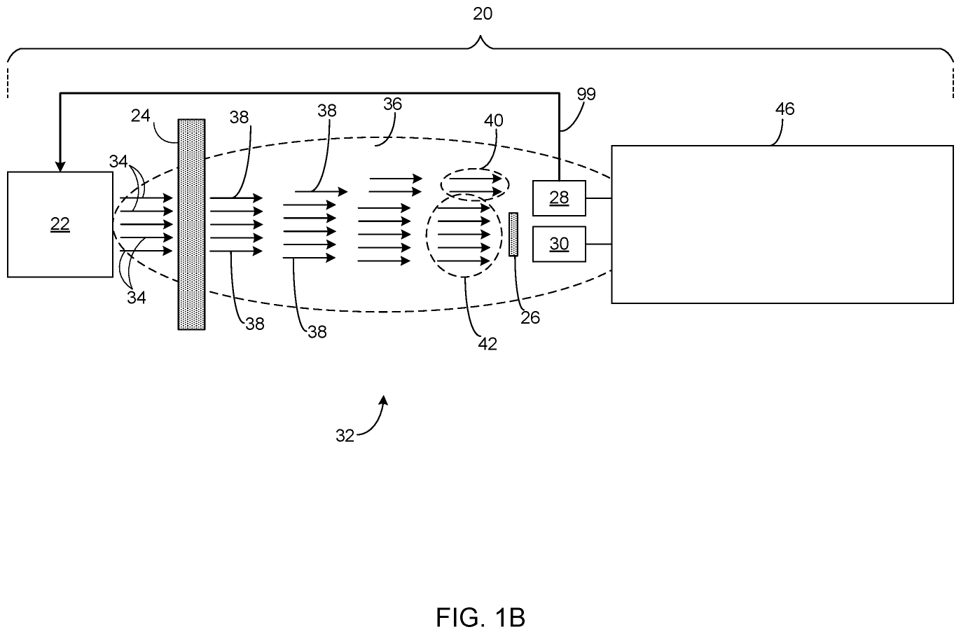

FIGS. 1A-B show an embodiment 20 of the invention, which comprises an optical energy source 22, a first polarizer 24, a second polarizer 26 spaced a distance from the first polarizer 24 having a rotation Θ relative to the first polarizer 24, a first detector 28, a second detector 30 collocated with the first detector 28, and a circuit 46. Each of the first detector 28 and second detector 30 is oriented to receive optical energy passing through a space 32. In the preferred embodiment, the first detector 28 and second detector 30 are silicon detectors. As used herein, “collocated” means being positioned near each other, although the disclosure will discuss below how that even such near placement may provide for light from a common source not entering each of the detectors with approximately equal intensity due to variations in ear tissue, and further solutions to address the associated challenges will be disclosed.

In addition, although the embodiment discloses the use of silicon detectors, other types of detectors may be used (e.g., photoresistors). As shown in FIG. 1B, in various instances, a feedback circuit 99 interconnects the optical energy source 22 and the first detector 28, although in yet further instances, the feedback circuit 99 may interconnect the optical energy source 22 and the second detector 30. The feedback circuit 99 operates to adjust the source optical energy magnitude of the optical energy from the optical energy source 22 in response to the energy of the optical energy received at the first detector 28, or in yet further instances, the second detector 30.

When actuated, the optical energy source 22 produces initial optical energy 34 having an emission pattern 36. The optical energy source 22 is preferably a red light source, such as a red light-emitting diode (LED) or a laser, but may alternatively be near-infrared. Ultimately, the initial optical energy 34 must be of a wavelength that may be affected by the presence of sugar in the subject fluid while also passing through the other vessel in which the fluid is contained. The initial optical energy 34 from the optical energy source 22 has a magnitude termed the source optical energy magnitude.

The first polarizer 24 is positioned proximal to the optical energy source 22, such that the initial optical energy 34 passes through the first polarizer 24 and becomes polarized energy 38. The polarized energy 38 traverses the space 32 between the first polarizer 24 and second polarizer 26, where a first portion 40 of the polarized energy 38 is detected by a first detector 28 and a second portion 42 of the polarized energy 38 passes through a second polarizer 26 to the second detector 30. Notably, first detector 28 and second detector 30 are collocated, despite the proximity of second polarizer 26 to the second detector 30. Because the space 32 is empty in FIG. 1, the polarized energy 38 passing through the space 32 is not rotated by, for example, the presence of a sugar in a fluid.

Preferably, the first polarizer 24 and second polarizer 26 are a linearly-polarized film because such film is inexpensive compared to other available alternatives. Such film, however, is optimal for energy wavelengths in the visible spectrum. Other polarizers may be used, provided that the selected wavelength of the optical energy source 22 is chosen to optimally correspond. For example, an alternative polarizer may be wire-grid or holographic, which is optimally configured for use in the present invention with energy of near-infrared and infrared wavelengths.

Preferably, the difference in rotation between the first polarizer 24 and second polarizer 26 is forty-five degrees (or an integral multiple of forty-five degrees) plus the rotation caused by the baseline. In this optimal case, a change in concentration relative to the baseline at least initially moves along the most linear portion of a sine wave, which makes detecting the change in rotation easier compared to moving further away from where the slope of the wave is 1 and further towards where the slope is 0 (i.e., the crest and troughs of the sine wave). For example, when used with a baseline glucose concentration 100 mg/dL over a length of L, Θ equals 0.014 degrees. In this case, the rotation between the polarizers should be 45.014 degrees. The greater the change in concentration from the baseline, however, the more non-linear the correlation of the rotation to the change in concentration.

The first detector 28 and second detector 30 are electrically coupled to the circuit 46. The circuit 46 compares the relative intensity of light detected by the first detector 28 and second detector 30.

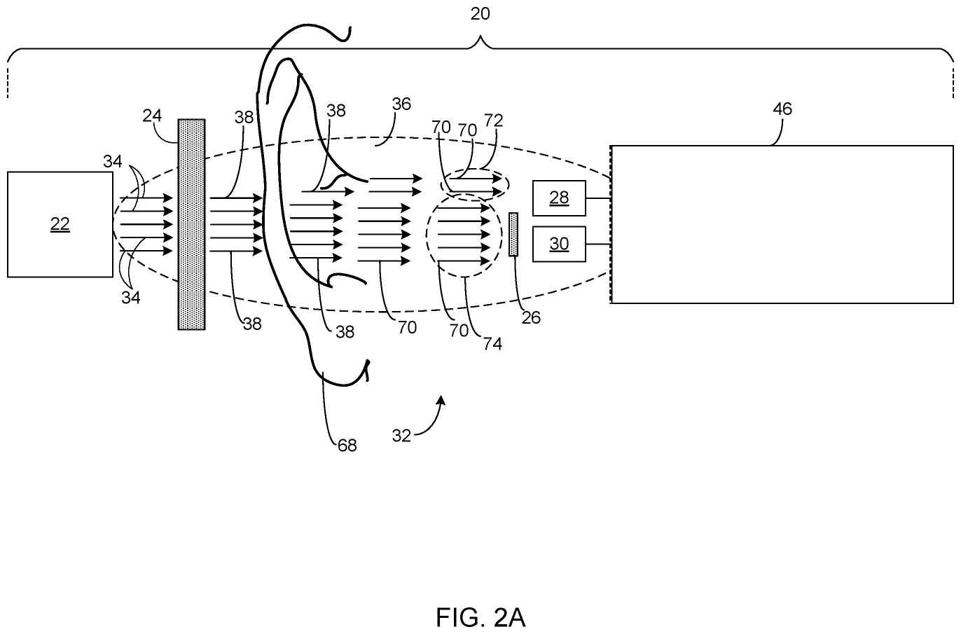

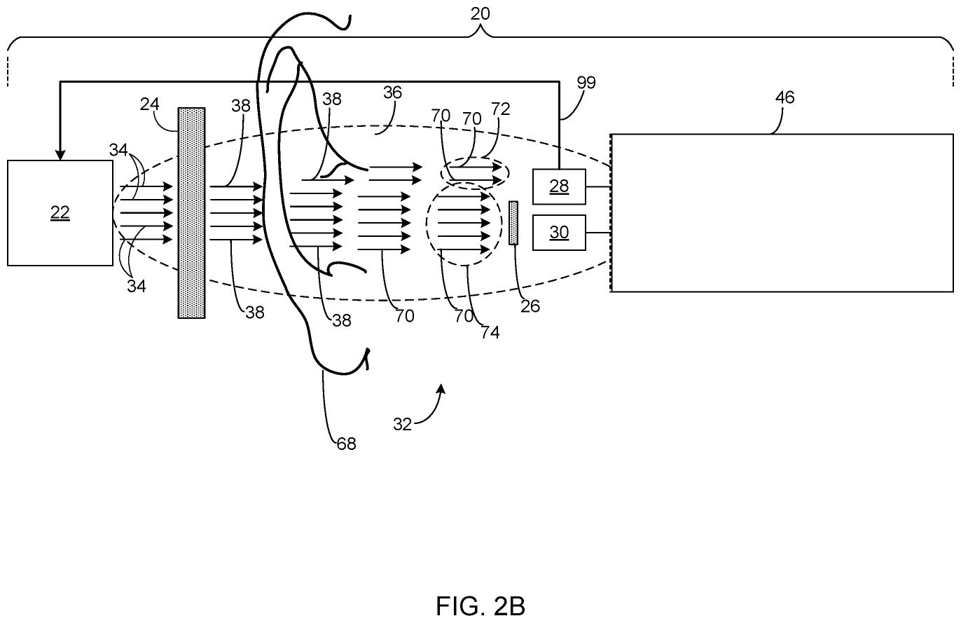

FIGS. 2A-B show the embodiment 20 in use with a human ear 68, at least a portion of which occupies the space 32. The preferred orientation of the human ear 68 within the space 32 is so that the polarized energy 38 passes through the human ear 68 generally parallel to a lateral axis, where L is the distance along the axis of the measured fluid. For most human ears, L is approximately three millimeters of capillary-rich and blood vessel-rich skin.

When actuated, the optical energy source 22 produces initial optical energy 34 having the emission pattern 36. The initial optical energy 34 passes through the first polarizer 24, and is of a wavelength to which the non-sugar components of the human ear 68 (i.e., skin, blood, tissue, cartilage) are, to at least some extent, transparent.

After passing through the first polarizer 24, the initial optical energy 34 becomes polarized energy 38. Glucose within the blood in the human ear 68, however, will cause a change in polarization of the polarized energy 38 according to Θ=α×L×C, causing the rotated energy 70 exiting the ear to have a first rotation Θ₁.

The intensity of a first portion 72 of the rotated energy 70 is detected by the first detector 28. The intensity of a second portion 74 of the rotated energy 70 passes through the second polarizer 26 and is detected by the second detector 30. Each of the first detector 28 and second detector 30 produces a signal representative of the received intensity. Because the intensity of the rotated energy 70 received by the second detector 30 is only the intensity of the rotated energy component passing through the second polarizer 26, by measuring the difference in intensities at the first detector 28 and second detector 30, the rotation caused by the glucose in the human ear 68 can be derived, from which the changed in glucose concentration relative to a baseline can be determined.

To determine the baseline, prior to use, the embodiment 20 is calibrated to a baseline glucose concentration of seventy mg/dL (a “normal” concentration for human blood) by changing a potentiometer to compensate for the difference in intensities of energy received by the first detector 28 and second detector 30. Thus, any change in measured rotation represents a change in glucose concentration from some baseline (e.g., 70 mg/dL).

An alternative embodiment of the invention is calibrated to a baseline glucose concentration of 100 mg/dL using wavelength of 650 nm, resulting in a rotation of 45.028 degrees of the second polarizer relative to the first polarizer. This results in a range of resulting rotation of the baseline plus or minus 0.2 degrees for a glucose concentration of between 30 mg/dL and 300 mg/dL. Thus, a glucose concentration of 30 mg/dL will result in a rotational difference between the detectors of 0.0096 degrees, whereas a glucose concentration of 300 mg/dL will result in a rotational difference of 0.0273 degrees in the opposite direction of the direction of the 30 mg/dL concentration.

With specific reference to FIG. 2B, notably, and differently from the discussion with reference to FIG. 2A above, a feedback circuit 99 conveys a feedback signal from first detector 28 to the optical energy source 22 producing initial optical energy 34. The feedback circuit 99 adjusts the optical energy source 22 to maintain the first portion 72 of the rotated energy within a first portion calibration range. More specifically, in response to the energy of the intensity of the first portion 72 of the rotated energy 70 deviating below a lower calibration threshold from a first target intensity value, the feedback signal directs the optical energy source 22 to increase the intensity of the initial optical energy 34 (source optical energy magnitude) until the intensity of the first portion 72 of the rotated energy 70 no longer falls below a lower calibration threshold from a first target intensity value. Similarly, in response to the intensity of the first portion 72 of the rotated energy 70 deviating above an upper calibration threshold from a first target intensity value, the feedback signal directs the optical energy source 22 to decrease the intensity of the initial optical energy 34 (source optical energy magnitude) until the intensity of the first portion 72 of the rotated energy 70 no longer is above the upper calibration threshold from the first target intensity value. The upper calibration threshold and the lower calibration threshold define the boundaries of the first portion calibration range.

While the difference between the intensity of the first portion 72 and the second portion 74 of the rotated energy is measured similarly to as discussed above, the intensity of the first portion 72 is maintained between the upper calibration threshold and the lower calibration threshold about the first target intensity value. Because the optical transmissivity of the human ear 68 changes exponentially with the tissue thickness, and yet the difference in intensity of the first portion 72 and second portion 74 relates to the glucose concentration according to a linear approximation, relatively small changes in tissue thickness can result in relatively large shifts along a numerical approximation curve, causing calculation errors. Consequently the feedback mechanism discussed herein maintains the comparison within the same or similar linear region of the approximation curve, aiding calculation accuracy.

As previously mentioned, to determine the baseline, prior to use, the embodiment 20 is calibrated to a baseline glucose concentration of seventy mg/dL (a “normal” concentration for human blood) by changing a potentiometer to compensate for the difference in intensities of energy received by the first detector 28 and second detector 30. Thus, any change in measured rotation represents a change in glucose concentration from some baseline (e.g., 70 mg/dL).

An alternative embodiment of the invention is calibrated to a baseline glucose concentration of 100 mg/dL using wavelength of 650 nm, resulting in a rotation of 45.028 degrees of the second polarizer relative to the first polarizer. This results in a range of resulting rotation of the baseline plus or minus 0.2 degrees for a glucose concentration of between 30 mg/dL and 300 mg/dL. Thus, a glucose concentration of 30 mg/dL will result in a rotational difference between the detectors of 0.0096 degrees, whereas a glucose concentration of 300 mg/dL will result in a rotational difference of 0.0273 degrees in the opposite direction of the direction of the 30 mg/dL concentration.

Notably, in various instances the feedback circuit 99 operates so that the determined baseline may be further adjusted to compensate for variations in the intensity of the first portion 72 of the rotated energy 70 detected by the first detector 28 and/or the intensity of the second portion 74 of the rotated energy 70 passed through the second polarizer 26 and detected by the second detector 30. For instance, variations in placement of the human ear 68 at least a portion of which occupies the space 32 may cause variations in the intensity of the first portion 72 and/or the intensity of the second portion 74. As such, in various instances, a feedback circuit 99 of the circuit 46 may cause the intensity of the first portion 72 or the intensity of the second portion 74 to be returned to at or near the determined baseline regardless of the relative inconsistency of positioning on the human ear 68. As a result, the rotation caused by the glucose in the human ear 68 can be derived. As mentioned, because the intensity of the rotated energy 70 received by the second detector 30 is only the intensity of the rotated energy component passing through the second polarizer 26, by measuring the difference in intensities at the first detector 28 and second detector 30, the rotation caused by the glucose in the human ear 68 can be derived, from which the changed in glucose concentration relative to a baseline can be determined.

To compensate for the difference in intensities of energy received by first detector 28 and second detector 30 to calibrate the embodiment 20 to a baseline glucose concentration of seventy mg/dL (a “normal” concentration for human blood), the device may actively implement feedback via the feedback circuit 99 to continuously or intermittently recalibrate so that any change in measured rotation represents a change in glucose concentration from some baseline (e.g., 70 mg/dL). By controlling the feedback circuit 99, the circuit 46 may learn compensation offset values and may store these values in a memory rather than requiring the changing of a potentiometer. In this manner the feedback circuit 99 may operate to account for circuit variations and allow recalibration of the relationship between measured rotation and change in glucose concentration from a base line. In this manner, the feedback circuit 99 may operate so that the slope intercept calculations may remain unhampered by the exponential effect on photon transmissivity of the human ear 68 (and associated exponential effect on intensity of detected light) that is caused by a linear change in a thickness of human ear 68. Thus the feedback circuit 99 may be multipurpose.

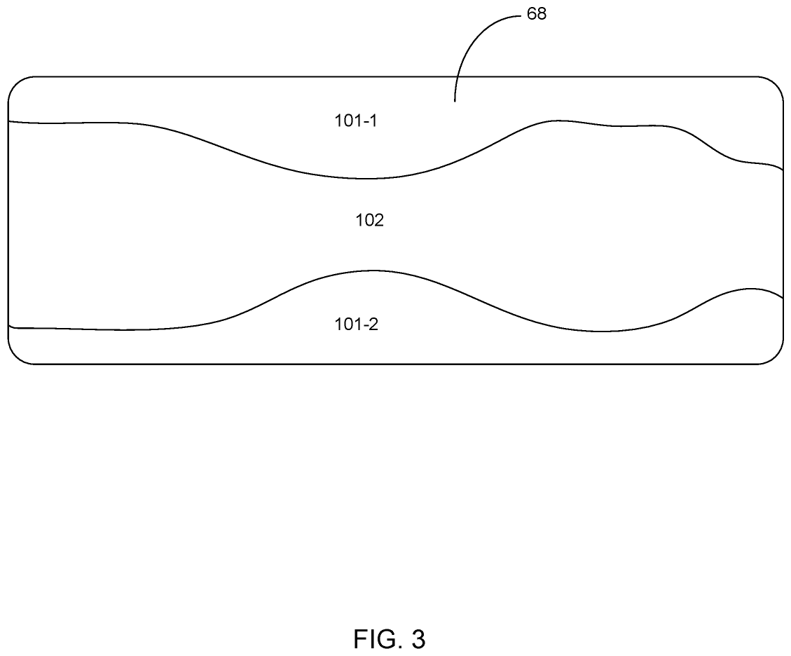

In various instances, however, particular challenges arise. For example, even despite close placement of the first detector 28 and second detector 30, the optical path from the source of optical energy to first detector 28 and second detector 30 may be sufficiently different to cause appreciable path differences to exist between the optical path of energy to the first detector 28 versus the second detector 30, contributing to unwanted results. For example, with reference to FIG. 3, an optical tissue model of a human ear 68 is provided. In various instances, a human ear 68 comprises a first outer tissue region 101-1 comprising a tissue of a first type, and a second outer tissue region 101-2 also comprising a tissue of a first type. The ear may also have an inner tissue region 102 comprising a tissue of second type.

In various instances the first outer tissue region 101-1 and the second outer tissue region 101-2 comprise blood rich tissues while the inner tissue region 102 may comprise a blood poor tissue. In various instances, the inner tissue region 102 is sandwiched between the first outer tissue region 101-1 and the second outer tissue region 101-2. Thus, one may say that the blood poor tissue is interstitial of the blood rich tissue.

One may appreciate that, as shown in FIG. 3, in various areas of the ear, the first outer tissue region 101-1 comprises a certain thickness, as does the second outer tissue region 101-2 and the inner tissue region 102. Each of the different tissue regions each has its own thickness. Moreover, at different locations on the ear, the different tissue regions each have different absolute thicknesses, as well as different relative thicknesses as compared one to another. Thus, the optical path for optical energy has different characteristics at different entry and exit locations on the ear due to these variations in tissue thickness Similarly, differences in tissue density may arise from place to place, causing further variations in optical path.

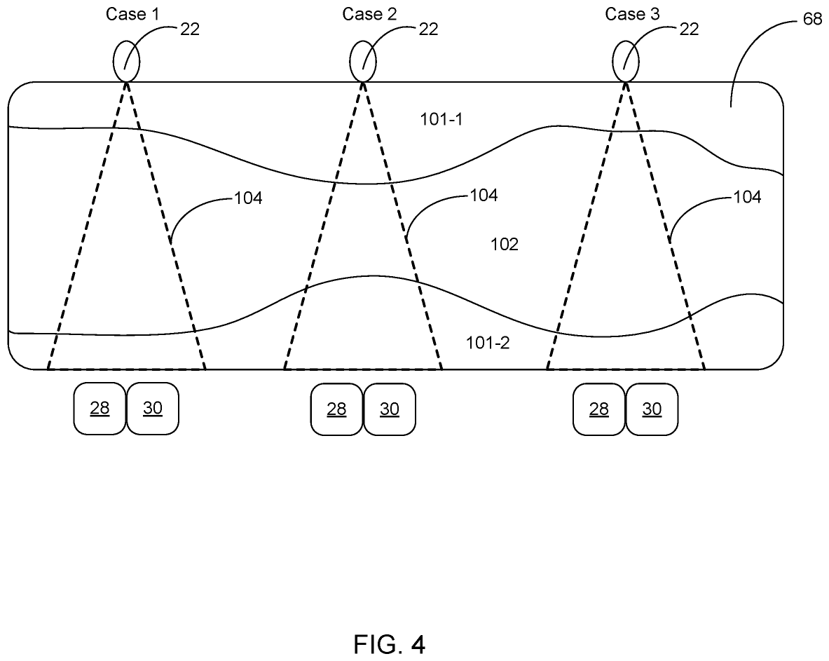

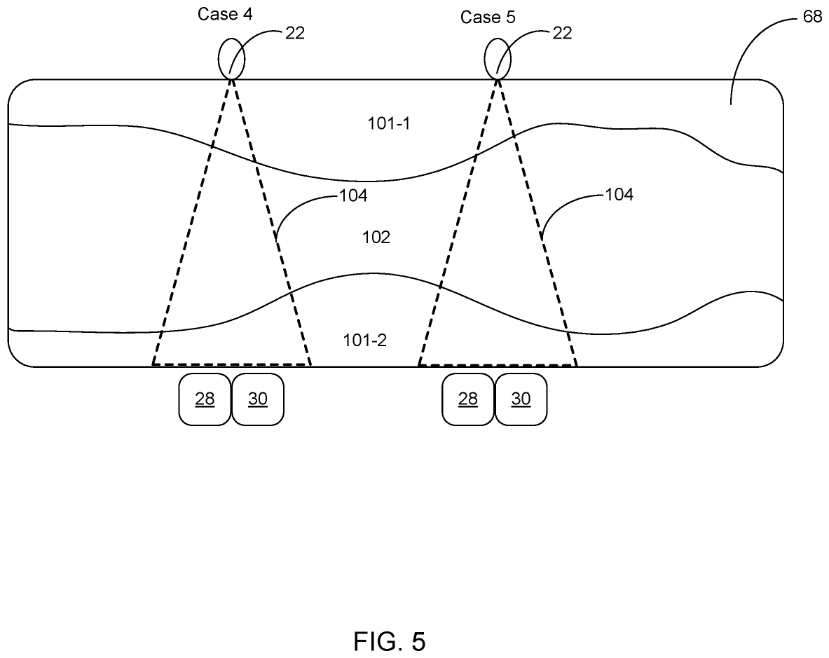

With reference to FIG. 4, three use cases are depicted. In case 1, an optical energy source 22 transmits optical energy along an optical path field 104. An optical path field 104 comprises a region that is illuminated by the optical energy source 22. Detectors placed at locations within the optical path field 104 may detect the optical energy. Because the optical path field 104 has sufficient width to illuminate both the first detector 28 and the second detector 30, there are different portions of the optical energy taking different paths through the ear within the optical path field 104. For instance, the optical energy traveling from the optical energy source 22 through the optical path field 104 to the first detector 28 may travel along a slightly different path than the optical energy traveling from the optical energy source 22 through the optical path field 104 to the second detector 30, Thus, with momentary reference to FIGS. 5-7, in addition to FIG. 4, the optical energy traveling to each detector may encounter different tissues having different thicknesses and/or different densities.

As shown in FIG. 4, cases 1, 2, and 3 each show a different potential optical path field 104. Notably however, within case 1, the thickness of the first outer tissue region 101-1 is relatively constant throughout the optical path field 104, the thickness of the second outer tissue region 101-2 is relatively constant throughout the optical path field 104, and the thickness of the inner tissue region 102 is relatively constant throughout the optical path field 104.

With reference to case 2 of FIG. 4, it is apparent that the thickness of the first outer tissue region 101-1 and the second outer tissue region 101-2 is relatively greater than in case 1, and the thickness of the inner tissue region 102 is relatively lesser than in case 1, but again, the thickness of the first outer tissue region 101-1 is relatively constant throughout the optical path field 104, the thickness of the second outer tissue region 101-2 is relatively constant throughout the optical path field 104, and the thickness of the inner tissue region 102 is relatively constant throughout the optical path field 104.

With reference to case 3 of FIG. 4, it is apparent again, that while there are some localized variations in thickness of the different tissue regions within the optical path field 104, once again, the thickness of the first outer tissue region 101-1 is relatively constant throughout the optical path field 104, the thickness of the second outer tissue region 101-2 is relatively constant throughout the optical path field 104, and the thickness of the inner tissue region 102 is relatively constant throughout the optical path field 104.

As a consequence, in case 1, of FIG. 4, the optical energy from optical energy source 22 arrives at both the first detector 28 and the second detector 30, having passed through relatively similar amounts of blood rich material and blood poor material on its travel through the ear material.

In case 2 of FIG. 4, the optical energy from optical energy source 22 arrives at both the first detector 28 and the second detector 30, having passed through relatively similar amounts of blood rich material and blood poor material on its travel through the ear material. Notably, a greater portion of the length of travel of the optical energy was through blood rich material than through blood poor, so a calibration may be necessary if the previous calibration was for a case 1 scenario wherein the ratio of blood rich to blood poor material was different. Alternatively, use of feedback via a feedback system as discussed, may effectuate an on-the-fly calibration. Notably though, the first detector 28 and the second detector 30 are relatively identically affected by the difference from case 1 to case 2, so that the path of the optical energy impinging each detector is relatively similar within case 2.

Likewise, in case 3 of FIG. 4, the optical energy from optical energy source 22 arrives at both the first detector 28 and the second detector 30 having passed through relatively similar amounts of blood rich material and blood poor material on their travel through the ear material.

Thus, it may be said that FIG. 4 depicts three cases, a case 1, case 2, and case 3 in which the first detector 28 and the second detector 30 enjoy matched optical paths, relative to each other.

In further instances, such as with reference to FIG. 5, the first detector 28 and the second detector 30 do not enjoy matched optical paths. For example, in case 4, the optical energy from optical energy source 22 arrives at the first detector 28 having passed through relatively less blood rich material and relatively more blood poor material, than the optical energy from optical energy source 22 arriving at the second detector 30. There are significant variations in the thickness of the first outer tissue region 101-1, the second outer tissue region 101-2, and the inner tissue region 102 at different optical paths throughout the scope of the optical path field 104. Also, while tissue thickness is depicted in the Figures, one having ordinary skill in the art will also understand that the discussion of variations in tissue thicknesses is also applicable to instances of variation in tissue density, opacity, and/or the like.

Similarly, with reference to case 5 of FIG. 5, the optical energy from optical energy source 22 arrives at the first detector 28 having passed through relatively more blood rich material and relatively less blood poor material, than the optical energy from optical energy source 22 arriving at the second detector 30. Thus, again the significant variations in the thickness of the first outer tissue region 101-1, the second outer tissue region 101-2 and the inner tissue region 102 at the different optical paths through the scope of the optical path field 104 affect the performance of the sensor system and method, due to the dissimilarity in path for optical energy impinging the first detector 28 and the second detector 30.

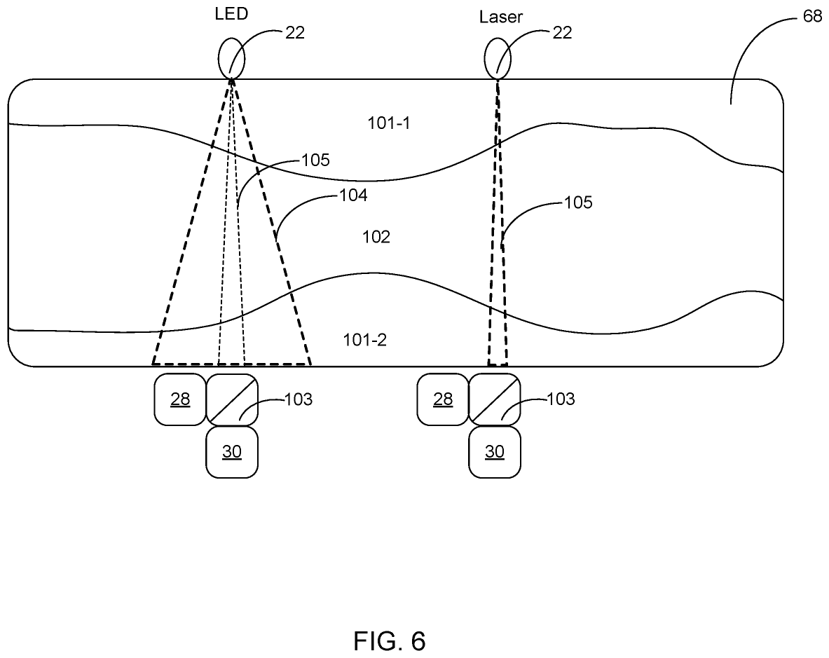

With reference to FIG. 6, various embodiments contemplate one response to the challenges discussed with reference to FIGS. 4 and 5. For example, an optical energy source 22 is provided. The optical source may comprise a non-collimated light source, such as an LED. The LED may illuminate the ear tissue, for instance, the first outer tissue region 101-1, the second outer tissue region 101-2, and the inner tissue region 102 with optical energy occupying an optical path field 104. Moreover, there may be significant variations in the optical paths at different points in the optical path field 104, such as due to variations in the densities, opacities, and/or thicknesses, both absolute and relative, of the first outer tissue region 101-1, the second outer tissue region 101-2, and the inner tissue region 102.

Thus, in various embodiments, rather than associating separate optical paths with the optical energy source 22 in relation to the first detector 28 and with the optical energy source 22 in relation to the second detector 30, in various embodiments, a detector beam splitter/combiner 103 may be implemented. For example, an optical energy source 22 may be proximate to one side of an ear, such as an outer surface of a first outer tissue region 101-1, and a detector beam splitter/combiner 103 may be proximate to an opposite side of an ear, such as an outer surface of a second outer tissue region 101-2. Thus, optical energy may pass along a single path and/or a narrowed path 105 within the optical path field 104, from the optical energy source 22 to the detector beam splitter/combiner 103. Because narrowed path 105 is much narrower than the optical path field 104, the variation in the thickness (both relative and absolute) of the first outer tissue region 101-1, the second outer tissue region 101-2 and the inner tissue region 102 across the narrowed path 105 is ameliorated. The first detector 28 and the second detector 30 are optically coupled to the detector beam splitter/combiner 103 and both receive optical energy from the optical energy source 22 that has passed through the narrowed path 105. In various embodiments, the narrowed path 105 is too narrow to facilitate illumination of adjacent first detector 28 and second detector 30. In that manner, the variations in the characteristics of the optical path between the optical energy source 22 and the first detector 28 and second detector 30 is ameliorated because each of the first detector 28 and second detector 30 receive illumination that has travelled along the same optical path to the detector beam splitter/combiner 103.

Moreover, in further embodiments, the optical source may comprise a collimated light source such as a laser. In various instances, a collimated light source such as a laser illuminates a narrowed path 105 rather than the wide optical path field 104, at least in part due to the effects of collimation.

In various embodiments, a laser may illuminate the ear tissue, for instance, the first outer tissue region 101-1, the second outer tissue region 101-2, and the inner tissue region 102 with optical energy occupying a narrowed path 105. Because there may be significant variations in the optical paths at different points proximate to the narrowed path 105, use of a collimated light source may narrow the illuminated area, so that only a narrow portion of the ear is illuminated. In various instances, the narrowness of the illuminated portion of the first outer tissue region 101-1, the second outer tissue region 101-2, and the inner tissue region 102 renders insufficiently broad area of illumination to permit the first detector 28 and second detector 30 to both be placed in a position to receive optical energy along the narrowed path 105. Moreover if the path were broad enough to illuminate both the first detector 28 and the second detector 30, then variations in the thicknesses, both absolute and relative, of the first outer tissue region 101-1, the second outer tissue region 101-2, and the inner tissue region 102 would cause the optical illumination to have different characteristics at the first detector 28 and the second detector 30 due to variations in absolute and/or relative opacity, density, and/or thickness of the different tissues. Thus, in various embodiments, rather than associating separate optical paths with the optical energy source 22 in relation to the first detector 28 and with the optical energy source 22 in relation to the second detector 30, a collimated light source generating optical energy along a narrowed path 105 may be implemented in connection with a detector beam splitter/combiner 103.

For example, an optical energy source 22 comprising a laser may be proximate to one side of an ear, such as an outer surface of a first outer tissue region 101-1, and a detector beam splitter/combiner 103 may be proximate to an opposite side of an ear, such as an outer surface of a second outer tissue region 101-2. Thus, optical energy may pass along a single path and/or a narrowed path 105, from the optical energy source 22 to the detector beam splitter/combiner 103. Because narrowed path 105 is sufficiently narrow, the variation in the density, opacity, and/or thickness (both relative and absolute) of the first outer tissue region 101-1, the second outer tissue region 101-2, and the inner tissue region 102 across the narrowed path 105 is ameliorated. The first detector 28 and the second detector 30 are optically coupled to the detector beam splitter/combiner 103 and both receive optical energy from the optical energy source 22 that has passed through the narrowed path 105, wherein the narrowed path 105 is too narrow to facilitate illumination of adjacent first detector 28 and second detector 30. In that manner, the variations in the characteristics of the optical path between the optical energy source 22 and first detector 28 and second detector 30 is ameliorated because each of the first detector 28 and second detector 30 receive illumination that has travelled along the same optical path to the detector beam splitter/combiner 103.

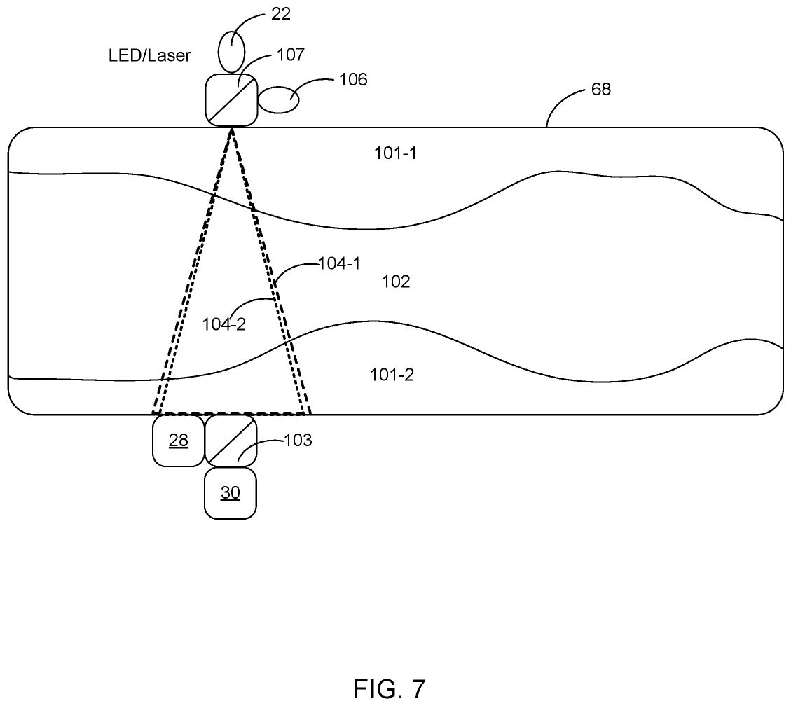

Finally, with reference to FIG. 7, further aspects are disclosed. In various instances, there may be a desire to combine multiple different types of optical energy sources. However, just as placing a first detector 28 and second detector 30 adjacently against the ear causes some path variation in optical energy directed to the first detector 28 versus optical energy to the second detector 30, placing different optical energy sources adjacently may cause path variations in optical energy originating from a first optical energy source versus that originating from a second optical energy source. For example, in various embodiments, optical energy source 22 operates in conjunction with filters to provide a polarized light to the ear. However, in various instances, it may be useful to also use a non-polarized light to generate calibration data useful to calibrate a system as discussed herein. For instance, variations in placement relative to an ear may result variations in characteristics associated with the energy as it passes through ear tissue of different opacity, density, and/or thickness. For instance, variations may cause changes in attenuation of the optical energy. A measurement of a non-polarized light source may be useful in characterizing these variations. For instance, the first outer tissue region 101-1, the second outer tissue region 101-2, and/or the inner tissue region 102 may have different attenuative properties at different locations. Thus, it may be useful to also pass a non-polarized light through the ear tissue to measure these relative attenuative properties.

In various embodiments, a non-polarized optical energy source 106 is combined with the optical energy source 22 via a source beam splitter/combiner 107. In various instances, the non-polarized optical energy source 106 comprises infrared or near-infrared light that passes through the same polarizing filters as the light of optical energy source 22, but due to its longer wavelength, does not experience significant polarizing effects from the polarization filters as it passes through.

Thus, an optical energy source 22 and a non-polarized optical energy source 106 may both illuminate a source beam splitter/combiner 107. The source beam splitter/combiner 107 may cause light from each source to illuminate an ear along an optical path field 104. The first optical path field 104-1 associated with the optical energy source 22 and the second optical path field 104-2 associated with the non-polarized optical energy source 106 may be coincident, or substantially coincident, such as due to their origination from the same source beam splitter/combiner 107, though some slight variation may arise. For example slight variation may arise due to wavelength-dependent variation in refraction. In various instances, while both the first optical path field 104-1 and second optical path field 104-2 are shown as relatively coincident, in various instances, one or both may correspond to a narrowed path 105 (similar to as shown in FIG. 6).

Both the first detector 28 and the second detector 30 are associated with a detector beam splitter/combiner 103. Thus, the optical illumination provided from source beam splitter/combiner 107 from either the optical energy source 22 or the non-polarized optical energy source 106 travels along a substantially same path to the detector beam splitter/combiner 103. From the detector beam splitter/combiner 103, the optical energy may be provided to the first detector 28 and the second detector 30, as discussed elsewhere herein.

Moreover, in various embodiments the optical energy source 22 and the non-polarized optical energy source 106 may be time division multiplexed and transmit optical energy at different times, or frequency division multiplexed and may transmit optical energy concurrently. Alternatively, one of the optical energy source 22 and the non-polarized optical energy source 106 may be pulsed while the other is not pulsed. Furthermore, the optical energy source 22 and/or the non-polarized optical energy source 106 may be modulated for later electronic isolation, as desired. In various embodiments, the non-polarized optical energy source 106 is duty cycled such as to provide an optical depth density measurement, which may be used to provide feedback to a user regarding whether the placement of the device relative to the ear is a “good” or “bad” placement for proper functioning of the system and method and/or further may facilitate software compensation of placement of the device in non-ideal locations. In various embodiments, one or both of the optical energy source 22 and non-polarized optical energy source 106 may be AC-coupled and/or encoded, such as to ameliorate noise.

Furthermore, while beams splitters are depicted at one or both ends of various illumination paths, the use of beam forming, such as by lenses and/or use of collimated light may replace the use of one or more beam splitter/combiner in various embodiments.

Thus one may appreciate that the source of optical energy emission may be adjusted to vary an intensity of the optical energy such as to compensate for varying optical thickness, density, and/or opacity occurring uniformly or non-uniformly across an optical path field 104 or a narrowed path 105. Moreover, the reduction in the detector footprint associated with the implementation of beam splitters, collimated light, narrowed paths 105 and/or the like may alleviate variation from location to location and ear to ear.

As discussed herein, the implementation of beam splitters may be termed the implementation of a “folded optical arrangement,” in various embodiments.

As discussed herein, the implementation of a non-polarized optical energy source 106 may be termed the implementation of an “interrogator beam” such as to interrogate tissue(s) to ascertain physical properties, such as attenuation.

The present disclosure includes preferred or illustrative embodiments in which specific sensors and methods are described. Alternative embodiments of such sensors can be used in carrying out the invention as claimed and such alternative embodiments are limited only by the claims themselves. Other aspects and advantages of the present invention may be obtained from a study of this disclosure and the drawings, along with the appended claims.

1. A non-invasive system for measuring glucose comprising:
a first light source emitting a light capable of penetrating a body tissue;
a beam splitter/combiner to receive the light;
a first detector optically coupled to the beam splitter/combiner; and
a second detector optically coupled to the beam splitter/combiner,
wherein the first detector and the second detector are operated to measure glucose in the body tissue in response to the light.

2. The non-invasive system of claim 1, wherein the first detector and the second detector measure glucose in the body tissue by calculating a difference in amplitude of the light detected by the first detector and amplitude of the light detected by the second detector.

3. The non-invasive system of claim 1, wherein at least one of the first detector and the second detector includes a polarizer.

4. The non-invasive system of claim 1, further comprising:
a first polarizer proximal to the first light source for receiving at least a portion of the light emitted from the first light source and for providing a first polarized light; and
a second polarizer to receive at least a portion of the first polarized light following passage of the first polarized light through the body tissue and to provide a second polarized light,
wherein the first detector is positioned in a manner to detect at least a portion of the first polarized light via the beam splitter/combiner; and
wherein the second detector is positioned in a manner to detect at least a portion of the second polarized light via the beam splitter/combiner,
wherein at least one of the first detector and the second detector determines a relative intensity of at least one of the first polarized light and the second polarized light.

5. The non-invasive system of claim 4, further comprising:
a second light source emitting a second light capable of penetrating the body tissue, the first light source and the second light source both providing light to a second beam splitter/combiner,
the second beam splitter/combiner combining light from both the first light source and the second light source for provision to the body tissue.

6. The non-invasive system of claim 4, wherein the first light source comprises a source of collimated light.

7. The non-invasive system of claim 5, wherein the first light source comprises a source of collimated light and the second light source comprises a source of non-collimated light.

8. The non-invasive system of claim 5, wherein the first light source comprises a laser.

9. A non-invasive system for measuring glucose comprising:
a first light source emitting a first light;
a polarizer configured to receive the first light and polarize the first light, the polarizer emitting a second light capable of penetrating a body tissue, the second light comprising polarized first light;
a first beam splitter/combiner to receive the second light following penetration into, through, and out of the body tissue;
a first detector optically coupled to the first beam splitter/combiner to receive the second light;
a second polarizer optically coupled to the first beam splitter/combiner to receive the second light and emit a third light comprising a further polarized second light; and
a second detector optically coupled to the second polarizer to receive the third light;
wherein the first detector and the second detector are operated to measure glucose in the body tissue by comparing an intensity of the second light and an intensity of the third light.

10. The non-invasive system of claim 9, further comprising:
a second light source emitting a fourth light;
a second beam splitter/combiner to receive the second light and the fourth light and provide the second light and the fourth light to the body tissue for penetration into the body tissue;
wherein the first beam splitter/combiner further receives the fourth light following penetration into, through, and out of the body tissue.

11. The non-invasive system of claim 10, wherein the first detector optically coupled to the first beam splitter/combiner further receives the fourth light.

12. The non-invasive system of claim 10, wherein at least one of the second detector and the first detector performs a calibration in response to the fourth light.

13. The non-invasive system of claim 10, wherein the first light source and the second light source emit light simultaneously.

14. The non-invasive system of claim 10, wherein the first light source and the second light source emit light simultaneously, the first light source emitting the first light having a first center frequency and the second light source emitting the fourth light having a second frequency, the first center frequency and the second center frequency being different frequencies.

15. The non-invasive system of claim 10, wherein the first light source emits first light that is modulated by a first modulation and wherein at least one of the first detector and the second detector detects the first modulation.

16. The non-invasive system of claim 10, wherein the first light source emits collimated light and the second light source emits non-collimated light.

17. A method of non-invasive glucose measurement comprising:
providing a first light source emitting a light capable of penetrating a body tissue;
providing a beam splitter/combiner to receive the light;
providing a first detector optically coupled to the beam splitter/combiner; and
providing a second detector optically coupled to the beam splitter/combiner,
wherein the first detector and the second detector are operated to measure glucose in the body tissue in response to the light.

18. The method of claim 17, further comprising:
providing a first polarizer proximal to the first light source for receiving at least a portion of the light emitted from the first light source and for providing a first polarized light; and
providing a second polarizer to receive at least a portion of the first polarized light following passage of the first polarized light through the body tissue and to provide a second polarized light,
wherein the first detector is providing positioned in a manner to detect at least a portion of the first polarized light via the beam splitter/combiner; and
wherein the second detector is providing positioned in a manner to detect at least a portion of the second polarized light via the beam splitter/combiner,
wherein at least one of the first detector and the second detector determines a relative intensity of at least one of the first polarized light and the second polarized light.

19. The method of claim 17, wherein the method further includes providing a polarizer with at least one of the first detector and the second detector.

20. The method of claim 17, wherein the first detector and the second detector are operated to measure glucose in the body tissue by calculating a difference in amplitude of the light detected by the first detector and amplitude of the light detected by the second detector.
